SEQUENCE LISTING (15 sequences)

SEQ ID NO: 1 - RNA, 22 nt, Homo sapiens
agagguaguagguugcauaguu

SEQ ID NO: 2 - RNA, 22 nt, Homo sapiens
aacccguagauccgaacuugug

SEQ ID NO: 3 - RNA, 22 nt, Homo sapiens
ugagguaguaguuugugcuguu

SEQ ID NO: 4 - RNA, 22 nt, Homo sapiens
ugagaacugaauuccauggguu

SEQ ID NO: 5 - RNA, 22 nt, Homo sapiens
cagcagcaauucauguuuugaa

SEQ ID NO: 6 - RNA, 22 nt, Homo sapiens
uagcuuaucagacugauguuga

SEQ ID NO: 7 - RNA, 21 nt, Homo sapiens
aucacauugccagggauuucc

SEQ ID NO: 8 - RNA, 21 nt, Homo sapiens
aucacauugccagggauuacc

SEQ ID NO: 9 - RNA, 23 nt, Homo sapiens
uagcaccauuugaaaucaguguu

SEQ ID NO: 10 - DNA, 2469 bp, Homo sapiens
atgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtaacagtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctga

SEQ ID NO: 11 - DNA, 6641 bp, Homo sapiens
gccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagagcatcagaaccagaggcttggtcccacaggccacggaccaatggcctgcagccgtgacaacactcctgtcatattggagtccaaaacttgaattctgggttgaattttttaaaaatcaggtaccacttgatttcatatgggaaattgaagcaggaaatattgagggcttcttgatcacagaaaactcagaagagatagtaatgctcaggacaggagcggcagccccagaacaggccactcatttagaattctagtgtttcaaaacacttttgtgtgttgtatggtcaataacatttttcattactgatggtgtcattcacccattaggtaaacattcccttttaaatgtttgtttgttttttgagacaggatctcactctgttgccagggctgtagtgcagtggtgtgatcatagctcactgcaacctccacctcccaggctcaagcctcccgaatagctgggactacaggcgcacaccaccatccccggctaatttttgtattttttgtagagacggggttttgccatgttgccaaggctggtttcaaactcctggactcaagaaatccacccacctcagcctcccaaagtgctaggattacaggcatgagccactgcgcccagcccttataaatttttgtatagacattcctttggttggaagaatatttataggcaatacagtcaaagtttcaaaatagcatcacacaaaacatgtttataaatgaacaggatgtaatgtacatagatgacattaagaaaatttgtatgaaataatttagtcatcatgaaatatttagttgtcatataaaaacccactgtttgagaatgatgctactctgatctaatgaatgtgaacatgtagatgttttgtgtgtatttttttaaatgaaaactcaaaataagacaagtaatttgttgataaatatttttaaagataactcagcatgtttgtaaagcaggatacattttactaaaaggttcattggttccaatcacagctcataggtagagcaaagaaagggtggatggattgaaaagattagcctctgtctcggtggcaggttcccacctcgcaagcaattggaaacaaaacttttggggagttttattttgcattagggtgtgttttatgttaagcaaaacatactttagaaacaaatgaaaaaggcaattgaaaatcccagctatttcacctagatggaatagccaccctgagcagaactttgtgatgcttcattctgtggaattttgtgcttgctactgtatagtgcatgtggtgtaggttactctaactggttttgtcgacgtaaacatttaaagtgttatattttttataaaaatgtttatttttaatgatatgagaaaaattttgttaggccacaaaaacactgcactgtgaacattttagaaaaggtatgtcagactgggattaatgacagcatgattttcaatgactgtaaattgcgataaggaaatgtactgattgccaatacaccccaccctcattacatcatcaggacttgaagccaagggttaacccagcaagctacaaagagggtgtgtcacactgaaactcaatagttgagtttggctgttgttgcaggaaaatgattataactaaaagctctctgatagtgcagagacttaccagaagacacaaggaattgtactgaagagctattacaatccaaatattgccgtttcataaatgtaataagtaatactaattcacagagtattgtaaatggtggatgacaaaagaaaatctgctctgtggaaagaaagaactgtctctaccagggtcaagagcatgaacgcatcaatagaaagaactcggggaaacatcccatcaacaggactacacacttgtatatacattcttgagaacactgcaatgtgaaaatcacgtttgctatttataaacttgtccttagattaatgtgtctggacagattgtgggagtaagtgattcttctaagaattagatacttgtcactgcctatacctgcagctgaactgaatggtacttcgtatgttaatagttgttctgataaatcatgcaattaaagtaaagtgatgcaacatcttgtaaaaaaaaaaaaaaaaaaaa

SEQ ID NO: 12 - DNA, 1101 bp, Homo sapiens
ctctgcttccttacagcacccccacctgccagagctgatcctccctaggccctgcctaaccttgagttggcccccaatccctctggctgcagaagtccccttacccccaatgagaggaggggcaggaccagatcttttgagagctgagggttgagggcattgagccaacacacagatttgtcgcctctgtccccgaagacacctgcaccctccatgcggagccaagatggggaatggaactgaggaagattataactttgtcttcaaggtggtgctgatcggcgaatcaggtgtggggaagaccaatctactctcccgattcacgcgcaatgagttcagccacgacagccgcaccaccatcggggttgagttctccacccgcactgtgatgttgggcaccgctgctgtcaaggctcagatctgggacacagctggcctggagcggtaccgagccatcacctcggcgtactatcgtggtgcagtgggggccctcctggtgtttgacctaaccaagcaccagacctatgctgtggtggagcgatggctgaaggagctctatgaccatgctgaagccacgatcgtcgtcatgctcgtgggtaacaaaagtgacctcagccaggcccgggaagtgcccactgaggaggcccgaatgttcgctgaaaacaatggactgctcttcctggagacctcagccctggactctaccaatgttgagctagcctttgagactgtcctgaaagaaatctttgcgaaggtgtccaagcagagacagaacagcatccggaccaatgccatcactctgggcagtgcccaggctggacaggagcctggccctggggagaagagggcctgttgcatcagcctctgaccttggccagcaccacctgcccccactggctttttggtgccccttgtccccacttcagccccaggacctttccttgccctttggttccagatatcagactgttccctgttcacagcaccctcagggtcttaaggtcttcatgccctatcacaaatacctcttttatctgtccacccctcacagactaggaccctcaaataaagctgttttatatcaaaaaaaaaaaaaaaaaa

SEQ ID NO: 13 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 14 - DNA, 5189 bp, Homo sapiens
tctgggggctcggctttgccgcgctcgctgcacttgggcgagagctggaacgtggaccagagctcggatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctctcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttggatattcttgaaagtttatatttttataattttttcttacatcagatgtttctttgcagtggcttaatgtttgaaattattttgtggctttttttgtaaatattgaaatgtagcaataatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattctaataaaatgtacatatataaatcaaaaaaaaaaaaaaaa

SEQ ID NO: 15 - DNA, 5830 bp, Homo sapiens
actgagtcccgggaccccgggagagcggtcagtgtgtggtcgctgcgtttcctctgcctgcgccgggcatcacttgcgcgccgcagaaagtccgtctggcagcctggatatcctctcctaccggcacccgcagacgcccctgcagccgccggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacaccccaactcccggacatcacatgagaggtctgctcagattttgaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctcttttttcattcatttaaaaagcattatcatgcccctgctgcgggtctcaccatgggtttagaacaaagagcttcaagcaatggccccatcctcaaagaagtagcagtacctggggagctgacacttctgtaaaactagaagataaaccaggcaacgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttagctgtctccttgccaagccccaggaagaaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttga

(19) United States
(12) Patent Application Publication
(10) Pub. No.: US 2012/0095030 A1
(43) Pub. Date: Apr. 19, 2012

(54) METHODS AND KITS TO PREDICT THERAPEUTIC OUTCOME OF TYROSINE KINASE INHIBITORS

(75) Inventor: Glen Weiss, Phoenix, AZ (US)

(73) Assignee: THE TRANSLATIONAL GENOMICS RESEARCH INSTITUTE, Phoenix, AZ (US)

(21) Appl. No.: 13/264,969

(22) PCT Filed: Apr. 19, 2010
(86) PCT No.: PCT/US10/31558

§ 371 Date: Dec. 29, 2011

Related U.S. Application Data
(60) Provisional application No. 61/170,260, filed on Apr. 17, 2009.
(60) Provisional application No. 61/293,685, filed on Jan. 10, 2010.

(51) Int. Cl. A61K 31/517; A61P 35/00; C40B 40/06; A61K 31/404; C40B 30/04; C07H 21/00

(52) U.S. Cl. 514/266.22; 506/9; 536/23.1; 506/16; 514/266.4; 514/414

(57) ABSTRACT

Methods of using specific microRNA to identify subjects with non-small cell lung cancer likely or unlikely to respond to treatment with tyrosine kinase inhibitors such as erlotinib, sunitinib, or vandetanib; methods of treating subjects based on identification of said subjects as likely to respond to treatment with tyrosine kinase inhibitors; and kits that facilitate the performance of the methods are disclosed.

### CROSS REFERENCE TO RELATED APPLICATION

This Application claims the benefit of U.S. Provisional Application No. 61/170,260, filed 17 Apr., 2009 and of U.S. Provisional Application No. 61/293,685 filed 10 Jan. 2010.

### BACKGROUND OF THE INVENTION

While FDA approved drugs are approved as safe and effective for the population as a whole, the majority of drugs do not work in all patients to which they are administered. However at present, many drugs are administered to patients without a prediction of whether or not the drug will be effective in a particular patient. This results in higher than necessary health care costs and risk to patients. There is a need in the art to develop new patient selection methods that will match patients to the correct treatment.

Worldwide, lung cancer is the leading cause of cancer-related mortality in both men and women. Although current treatments for advanced non-small cell lung cancer (NSCLC) are disappointing, there is growing promise using tyrosine kinase inhibitors (TM) such as erlotinib, vandetanib, or sunitinib. However, such therapies are being administered to patients in an unselected fashion.

### BRIEF SUMMARY OF THE INVENTION

The present invention provides among other things, a personalized medicine based method that allows the selection of lung cancer patients most likely to respond or not to respond to one or more tyrosine kinase inhibitors.

It is an object of the invention to identify tumors that are resistant or sensitive to tyrosine kinase inhibitors.

It is an object of the invention to stage patients with regard to treatment with tyrosine kinase inhibitors, assuring that the treatment is more likely to be given to those patients with the best chance of responding to it.

It is an object of the invention to allow health care providers to select other treatments for patients for whom a particular tyrosine kinase inhibitor is unlikely to work.

It is an object of the invention to provide a test that allows the prediction of whether or not a non-small cell lung cancer patient will respond to a tyrosine kinase inhibitor.

It is an object of the invention to provide a test that allows a health care provider to select of a particular tyrosine kinase inhibitor or combination of inhibitors to treat a non-small cell lung cancer patient.

It is an object of the invention to classify subjects into cohorts that include individuals likely to respond to a particular tyrosine kinase inhibitor or individuals not likely to respond to a particular tyrosine kinase inhibitor.

It is an object of the invention to treat a subject on the basis of a result that indicates whether or not a patient will respond to a tyrosine kinase inhibitor.

It is an object of the invention to provide kits that facilitate the identification of a patient as likely to respond to a tyrosine kinase inhibitor or unlikely to respond to a tyrosine kinase inhibitor.

The above and other objects may be achieved through the use of methods involving receiving a sample from a subject and isolating RNA from the sample, adding a first reagent capable of specific binding to a marker that includes a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, and SEQ ID NO. 15 to a mixture comprising the sample and subjecting the mixture to conditions that allow detection of the binding of the first reagent to the marker. The subject is suspected of having non-small cell lung cancer, the cohort includes two or more individuals unlikely to respond to treatment with a tyrosine kinase inhibitor, and the tyrosine kinase inhibitor is selected from the group consisting of erlotinib, sunitinib, and vandetanib. The first reagent may comprise a first oligonucleotide such as a stem-loop oligonucleotide. The method may further comprise adding reverse transcriptase and subjecting the mixture to conditions that comprise allowing the formation of a DNA template comprising the marker. The method may further comprise adding a second oligonucleotide and a third oligonucleotide to the mixture. The second oligonucleotide and the third oligonucleotide bind to opposite strands of the DNA template. For example, if the second oligonucleotide binds to the 5′→3′ strand, then the third oligonucleotide binds to the 3′→5′ strand. The method may further comprise adding a fourth oligonucleotide to the mixture. The fourth oligonucleotide binds to the DNA template between the sequences to which the second oligonucleotide and the third oligonucleotide are capable of binding. The fourth oligonucleotide may comprise a label. The label may be any label including a fluorescent label such as FAM, dR110, 5-FAM, 6FAM, dR6G, JOE, HEX, VIC, TET, dTAMRA, TAMRA, NED, dROX, PET, BHQ, Gold540, or LIZ. Alternatively, the conditions may comprise DNA sequencing. The first reagent may be affixed to a substrate. The sample may be any sample including a sample comprising serum or a sample comprising one or more cells such as a lung biopsy or metastatic tumor. The method may also include collecting the sample from the subject. If the marker includes a sequence selected from SEQ ID NO. 1, SEQ ID NO. 2, SEQ ID NO. 3, SEQ ID NO. 4, SEQ ID NO. 6, SEQ ID NO. 10, SEQ ID NO. 11, SEQ ID NO. 12, SEQ ID NO. 13, SEQ ID NO. 14, and SEQ ID NO. 15, then the tyrosine kinase inhibitor may comprise erlotinib. If the marker includes a sequence selected from SEQ ID NO. 5, SEQ ID NO. 6, SEQ ID NO. 7, SEQ ID NO. 8, and SEQ ID NO. 9, then the tyrosine kinase inhibitor may comprise sunitinib. If the marker includes SEQ ID NO. 6, then the tyrosine kinase inhibitor may comprise vandetanib.

The above and other objects may be achieved through the use of kits comprising a first reagent capable of specific binding to a marker that includes a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, and SEQ ID NO. 15 and an indication of a result that signifies classification of the subject into a cohort, wherein the cohort comprises two or more individuals unlikely to respond to treatment with a tyrosine kinase inhibitor and wherein the tyrosine kinase inhibitor is selected from the group consisting of erlotinib, sunitinib, and vandetanib. The first reagent may comprise a first oligonucleotide such as a stem loop oligonucleotide. The kit may further comprise a second oligonucleotide and a third oligonucleotide wherein the second oligonucleotide and the third oligonucleotide are capable of binding to opposite strands of a DNA construct comprising the reverse transcription product of the marker. For example, if the second oligonucleotide binds to the 5′→3′ strand, then the third oligonucleotide binds to the 3′→5′ strand. The kit may further comprise a fourth oligonucleotide capable of binding to a sequence between the sequences to which the second oligonucleotide and the third oligonucleotide are capable of binding. The fourth oligonucleotide may comprise a label, including a fluorescent label such as FAM, dR110, 5-FAM, 6FAM, dR6G, JOE, HEX, VIC, TET, dTAMRA, TAMRA, NED, dROX, PET, or LIZ. The kit may also comprise an enzyme such as a DNA polymerase, (including, for example, a thermostable DNA polymerase) or a reverse transcriptase. Alternatively, the first reagent may be affixed to a substrate. The kit may further comprise a device to be used in collecting a sample. The result may comprise a ΔCt value. The result may alternatively comprise a nucleic acid sequence listing. The indication may comprise a positive control. Alternatively, the indication may comprise a writing that may be physically included in the kit, may be made available via a website, may comprise an amplification plot, or may comprise a photograph. Alternatively, the indication may comprise software configured to detect the result as input and classification of the subject into the cohort as output. The software may be incorporated into any machine including a machine configured to detect fluorescence.

The above and other objects may be achieved through the use of methods involving receiving a sample from a subject and isolating RNA from the sample, adding a first reagent capable of specific binding to a marker that includes a sequence selected from SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, and SEQ ID NO. 15 to a mixture; subjecting the mixture to conditions that allow detection of the binding of the first reagent to the sequence, and treating with a tyrosine kinase inhibitor based upon a result indicated by the binding of the first reagent to the sequence. The subject is suspected of having non-small cell lung cancer. The cohort comprises two or more individuals likely to respond to treatment with a tyrosine kinase inhibitor. The tyrosine kinase inhibitor may be selected from erlotinib, sunitinib, or vandetanib. The first reagent may comprise a first oligonucleotide such as a stem loop oligonucleotide. The method may further comprise adding reverse transcriptase and subjecting the mixture to conditions that comprise allowing the formation of a DNA template comprising the marker. The method may further comprise adding a second oligonucleotide and a third oligonucleotide to the mixture. The second oligonucleotide and the third oligonucleotide bind to opposite strands of the DNA template. For example, if the second oligonucleotide binds to the 5′→3′ strand, then the third oligonucleotide binds to the 3′→5′ strand. The method may further comprise adding a fourth oligonucleotide to the mixture. The fourth oligonucleotide binds to the DNA template between the sequences to which the second oligonucleotide and the third oligonucleotide are capable of binding. The fourth oligonucleotide may comprise a label. The label may be any label including a fluorescent label such as FAM, dR110, 5-FAM, 6FAM, dR6G, JOE, HEX, VIC, TET, dTAMRA, TAMRA, NED, dROX, PET, BHQ, Gold540, or LIZ. Alternatively, the conditions may comprise DNA sequencing. The first reagent may be affixed to a substrate. The sample may be any sample including a sample comprising serum or a sample comprising one or more cells such as a lung biopsy or tumor sample. The method may also include collecting the sample from the subject. If the marker includes a sequence selected from SEQ ID NO. 1, SEQ ID NO. 2, SEQ ID NO. 3, SEQ ID NO. 4, SEQ ID NO. 6, SEQ ID NO. 10, SEQ ID NO. 11, SEQ ID NO. 12, SEQ ID NO. 13, SEQ ID NO. 14, or SEQ ID NO. 15 then the tyrosine kinase inhibitor may comprise erlotinib. If the marker includes a sequence selected from SEQ ID NO. 5, SEQ ID NO. 6, SEQ ID NO. 7, SEQ ID NO. 8, and SEQ ID NO. 9 then the tyrosine kinase inhibitor may comprise sunitinib. If the marker includes SEQ ID NO. 6, then the tyrosine kinase inhibitor may comprise vandetanib. Classifying the subject into a group may be performed on the recommendation of a writing. The writing may be affixed to a container housing the tyrosine kinase inhibitor. The result may be any result including a ΔCt value or nucleic acid sequence data.

### BRIEF DESCRIPTION OF THE FIGURES

A more complete understanding of the present invention may be derived by referring to the detailed description when considered in connection with the following illustrative figures.

FIG. 1 depicts the results of IC₅₀ determination of erlotinib sensitivity of HCC827 parent line (HCC827 NT) and the HCC827 erlotinib resistant line (HCC827 ER).

FIG. 2 depicts miRNA expression measured by qRT-PCR in HCC827 NT and HCC827 ER. Data were normalized to the ratio of hsa-miR-103 to hsa-miR-191. The * indicates a significant difference at p<0.05.

FIG. 3 depicts mRNA expression in HCC827 NT (gray) and HCC827 ER lines (black). Expression was normalized to the average expression of actin, GADPH, and histone H3.3. The * indicates a significant difference at p<0.05.

FIG. 4 depicts IC₅₀ of the H1703 parent line and H1703 lines treated to have sunitinib resistance (SRa, SRb, and SRc).

Elements and acts in the figures are illustrated for simplicity and have not necessarily been rendered according to any particular sequence or embodiment.

### DETAILED DESCRIPTION OF THE INVENTION

A marker may be any molecular structure produced by a cell, expressed inside the cell, accessible on the cell surface, or secreted by the cell. A marker may be any protein, carbohydrate, fat, nucleic acid, catalytic site, or any combination of these such as an enzyme, glycoprotein, cell membrane, virus, cell, organ, organelle, or any uni- or multimolecular structure or any other such structure now known or yet to be disclosed whether alone or in combination. A marker may also be called a target and the terms are used interchangeably.

A marker may be represented by the sequence of a nucleic acid from which it can be derived or any other chemical structure. Examples of such nucleic acids include miRNA, tRNA, siRNA, mRNA, cDNA, or genomic DNA sequences including complimentary sequences. Alternatively, a marker may be represented by a protein sequence. The concept of a marker is not limited to the products of the exact nucleic acid sequence or protein sequence by which it may be represented. Rather, a marker encompasses all molecules that may be detected by a method of assessing the expression of the marker.

When a nucleic acid includes a particular sequence, the sequence may be a part of a longer nucleic acid or may be the entirety of the sequence. The nucleic acid may contain nucleotides 5′ of the sequence, 3′ of the sequence, or both. The concept of a nucleic acid including a particular sequence further encompasses nucleic acids that contain less than the full sequence that are still capable of specifically detecting an allele. Nucleic acid sequences may be identified by the IUAPC letter code which is as follows: A—Adenine base; C—Cytosine base; G—guanine base; T or U—thymine or uracil base. M-A or C; R-A or G; W-A or T; S-C or G; Y-C or T; K-G or T; V-A or C or G; H-A or C or T; D-A or G or T; B-C or G or T; N or X-A or C or G or T. Note that T or U may be used interchangeably depending on whether the nucleic acid is DNA or RNA. A sequence having less than 60% 70%, 80%, 90%, 95%, 99% or 100% identity to the identifying sequence may still be encompassed by the invention if it is able of binding to its complimentary sequence and/or facilitating nucleic acid amplification of a desired target sequence. If a sequence is represented in degenerate form; for example through the use of codes other than A, C, G, T, or U; the concept of a nucleic acid including the sequence also encompasses a mixture of nucleic acids of different sequences that still meet the conditions imposed by the degenerate sequence.

Examples of molecules encompassed by a marker represented by a particular sequence or structure include point mutations, silent mutations, deletions, frameshift mutations, translocations, alternative splicing derivatives, differentially methylated sequences, differentially modified protein sequences, truncations, soluble forms of cell membrane associated markers, and any other variation that results in a product that may be identified as the marker. The following nonlimiting examples are included for the purposes of clarifying this concept: If expression of a specific marker in a sample is assessed by RTPCR, and if the sample expresses an mRNA sequence different from the sequence used to identify the specific marker by one or more nucleotides, but the marker may still be detected using RTPCR, then the specific marker encompasses the sequence present in the sample. Alternatively if expression of a specific marker in a sample is assessed by an antibody and the amino acid sequence of the marker in the sample differs from a sequence used to identify marker by one or more amino acids, but the antibody is still able to bind to the version of the marker in the sample, then the specific marker encompasses the sequence present in the sample.

Expression encompasses any and all processes through which material derived from a nucleic acid template may be produced. Expression thus includes processes such as RNA transcription, mRNA splicing, protein translation, protein folding, post-translational modification, membrane transport, associations with other molecules, addition of carbohydrate moeties to proteins, phosphorylation, protein complex formation and any other process along a continuum that results in biological material derived from genetic material whether in vitro, in vivo, or ex vivo. Expression also encompasses all processes through which the production of material derived from a nucleic acid template may be actively or passively suppressed. Such processes include all aspects of transcriptional and translational regulation. Examples include heterochromatic silencing, transcription factor inhibition, any form of RNAi silencing, microRNA silencing, alternative splicing, protease digestion, posttranslational modification, and alternative protein folding.

Expression may be assessed by any number of methods used to detect material derived from a nucleic acid template used currently in the art and yet to be developed. Examples of such methods include any nucleic acid detection method including the following nonlimiting examples, microarray analysis, RNA in situ hybridization, RNAse protection assay, Northern blot, reverse transcriptase PCR, quantitative PCR, quantitative reverse transcriptase PCR, quantitative real-time reverse transcriptase PCR, reverse transcriptase treatment followed by direct sequencing, direct sequencing of genomic DNA, or any other method of detecting a specific nucleic acid now known or yet to be disclosed. Other examples include any process of assessing protein expression including flow cytometry, immunohistochemistry, ELISA, Western blot, and immunoaffinity chromatography, HPLC, mass spectrometry, protein microarray analysis, PAGE analysis, isoelectric focusing, 2-D gel electrophoresis, or any enzymatic assay or any method that uses a protein reagent, nucleic acid reagent, or other reagent capable of specifically binding to or otherwise recognizing a specific nucleic acid or protein marker.

Other methods used to assess expression include the use of such natural or artificial ligands capable of specifically binding a marker. Such ligands include antibodies, antibody complexes, conjugates, natural ligands, small molecules, nanoparticles, or any other molecular entity capable of specific binding to a marker. Antibodies may be monoclonal, polyclonal, or any antibody fragment including an Fab, F(ab)₂, Fv, scFv, phage display antibody, peptibody, multispecific ligand, or any other reagent with specific binding to a marker. Ligands may be associated with a label such as a radioactive isotope or chelate thereof, dye (fluorescent or nonfluorescent,) stain, enzyme, metal, or any other substance capable of aiding a machine or a human eye from differentiating a cell expressing a marker from a cell not expressing a marker. Additionally, expression may be assessed by monomeric or multimeric ligands associated with substances capable of killing the cell. Such substances include protein or small molecule toxins, cytokines, pro-apoptotic substances, pore forming substances, radioactive isotopes, or any other substance capable of killing a cell.

Differential expression encompasses any detectable difference between the expression of a marker in one sample relative to the expression of the marker in another sample. Differential expression may be assessed by a detector, an instrument containing a detector, by aided or unaided human eye, or any other method that may detect differential expression. Examples include but are not limited to differential staining of cells in an IHC assay configured to detect a marker, differential detection of bound RNA on a microarray to which a sequence capable of binding to the marker is bound, differential results in measuring RTPCR measured in ΔCt or alternatively in the number of PCR cycles necessary to reach a particular optical density at a wavelength at which a double stranded DNA binding dye (e.g. SYBR Green) incorporates, differential results in measuring label from a reporter probe used in a real-time RTPCR reaction, differential detection of fluorescence on cells using a flow cytometer, differential intensities of bands in a Northern blot, differential intensities of bands in an RNAse protection assay, differential cell death measured by apoptotic markers, differential cell death measured by shrinkage of a tumor, or any method that allows a detection of a difference in signal between one sample or set of samples and another sample or set of samples.

The expression of the marker in a sample may be compared to a level of expression predetermined to predict the presence or absence of a particular cellular or physiological characteristic. The level of expression may be derived from a single control or a set of controls. A control may be any sample with a previously determined level of expression. A control may comprise material within the sample or material from sources other than the sample. Alternatively, the expression of a marker in a sample may be compared to a control that has a level of expression predetermined to signal or not signal a cellular or physiological characteristic. This level of expression may be derived from a single source of material including the sample itself or from a set of sources. Comparison of the expression of the marker in the sample to a particular level of expression results in a prediction that the sample exhibits or does not exhibit the cellular or physiological characteristic.

Prediction of a cellular or physiological characteristic includes the prediction of any cellular or physiological state that may be predicted by assessing the expression of a marker. Examples include the identity of a cell as a particular cell including a particular normal or cancer cell type, the likelihood that one or more diseases is present or absent, the likelihood that a present disease will progress, remain unchanged, or regress, the likelihood that a disease will respond or not respond to a particular therapy, or any other disease outcome. Further examples include the likelihood that a cell will move, senesce, apoptose, differentiate, metastasize, or change from any state to any other state or maintain its current state.

Expression of a marker in a sample may be more or less than that of a level predetermined to predict the presence or absence of a cellular or physiological characteristic. The expression of the marker in the sample may be more than 1,000,000×, 100,000×, 10,000×, 1000×, 100×, 10×, 5×, 2×, 1×, 0.5×, 0.1×0.01×, 0.001×, 0.0001×, 0.00001×, 0.000001×, 0.0000001× or less than that of a level predetermined to predict the presence or absence of a cellular or physiological characteristic.

The invention contemplates assessing the expression of the marker in any biological sample from which the expression may be assessed. One skilled in the art would know to select a particular biological sample and how to collect said sample depending upon the marker that is being assessed. Examples of sources of samples include but are not limited to biopsy or other in vivo or ex vivo analysis of prostate, breast, skin, muscle, facia, brain, endometrium, lung, head and neck, pancreas, small intestine, blood, liver, testes, ovaries, colon, skin, stomach, esophagus, spleen, lymph node, bone marrow, kidney, placenta, or fetus. In some aspects of the invention, the sample comprises a fluid sample, such as peripheral blood, lymph fluid, ascites, serous fluid, pleural effusion, sputum, cerebrospinal fluid, amniotic fluid, lacrimal fluid, stool, or urine. Samples include single cells, whole organs or any fraction of a whole organ, in any condition including in vitro, ex vivo, in vivo, post-mortem, fresh, fixed, or frozen.

One type of cellular or physiological characteristic is the risk that a particular disease outcome will occur. Assessing this risk includes the performing of any type of test, assay, examination, result, readout, or interpretation that correlates with an increased or decreased probability that an individual has had, currently has, or will develop a particular disease, disorder, symptom, syndrome, or any condition related to health or bodily state. Examples of disease outcomes include, but need not be limited to survival, death, progression of existing disease, remission of existing disease, initiation of onset of a disease in an otherwise disease-free subject, or the continued lack of disease in a subject in which there has been a remission of disease. Assessing the risk of a particular disease encompasses diagnosis in which the type of disease afflicting a subject is determined. Assessing the risk of a disease outcome also encompasses the concept of prognosis. A prognosis may be any assessment of the risk of disease outcome in an individual in which a particular disease has been diagnosed. Assessing the risk further encompasses prediction of therapeutic response in which a treatment regimen is chosen based on the assessment. Assessing the risk also encompasses a prediction of overall survival after diagnosis.

Determining the level of expression that signifies a physiological or cellular characteristic may be assessed by any of a number of methods. The skilled artisan will understand that numerous methods may be used to select a level of expression for a particular marker or a plurality of markers that signifies a particular physiological or cellular characteristic. In diagnosing the presence of a disease, a threshold value may be obtained by performing the assay method on samples obtained from a population of patients having a certain type of disease (cancer for example,) and from a second population of subjects that do not have the disease. In assessing disease outcome or the effect of treatment, a population of patients, all of which have, a disease such as cancer, may be followed for a period of time. After the period of time expires, the population may be divided into two or more groups. For example, the population may be divided into a first group of patients whose disease progresses to a particular endpoint and a second group of patients whose disease does not progress to the particular endpoint. Examples of endpoints include disease recurrence, death, metastasis or other states to which disease may progress. If expression of the marker in a sample is more similar to the predetermined expression of the marker in one group relative to the other group, the sample may be assigned a risk of having the same outcome as the patient group to which it is more similar.

In addition, one or more levels of expression of the marker may be selected that provide an acceptable ability of its ability to signify a particular physiological or cellular characteristic. Examples of such characteristics include identifying or diagnosing a particular disease, assessing a risk of outcome or a prognostic risk, or assessing the risk that a particular treatment will or will not be effective.

For example, Receiver Operating Characteristic curves, or “ROC” curves, may be calculated by plotting the value of a variable versus its relative frequency in two populations. For any particular marker, a distribution of marker expression levels for subjects with and without a disease may overlap. This indicates that the test does not absolutely distinguish between the two populations with complete accuracy. The area of overlap indicates where the test cannot distinguish the two groups. A threshold is selected. Expression of the marker in the sample above the threshold indicates the sample is similar to one group and expression of the marker below the threshold indicates the sample is similar to the other group. The area under the ROC curve is a measure of the probability that the expression correctly indicated the similarity of the sample to the proper group. See, e.g., Hanley et al., Radiology 143: 29-36 (1982) hereby incorporated by reference.

Additionally, levels of expression may be established by assessing the expression of a marker in a sample from one patient, assessing the expression of additional samples from the same patient obtained later in time, and comparing the expression of the marker from the later samples with the initial sample or samples. This method may be used in the case of markers that indicate, for example, progression or worsening of disease or lack of efficacy of a treatment regimen or remission of a disease or efficacy of a treatment regimen.

Other methods may be used to assess how accurately the expression of a marker signifies a particular physiological or cellular characteristic. Such methods include a positive likelihood ratio, negative likelihood ratio, odds ratio, and/or hazard ratio. In the case of a likelihood ratio, the likelihood that the expression of the marker would be found in a sample with a particular cellular or physiological characteristic is compared with the likelihood that the expression of the marker would be found in a sample lacking the particular cellular or physiological characteristic.

An odds ratio measures effect size and describes the amount of association or non-independence between two groups. An odds ratio is the ratio of the odds of a marker being expressed in one set of samples versus the odds of the marker being expressed in the other set of samples. An odds ratio of 1 indicates that the event or condition is equally likely to occur in both groups. An odds ratio grater or less than 1 indicates that expression of the marker is more likely to occur in one group or the other depending on how the odds ratio calculation was set up. A hazard ratio may be calculated by estimate of relative risk. Relative risk is the chance that a particular event will take place. It is a ratio of the probability that an event such as development or progression of a disease will occur in samples that exceed a threshold level of expression of a marker over the probability that the event will occur in samples that do not exceed a threshold level of expression of a marker. Alternatively, a hazard ratio may be calculated by the limit of the number of events per unit time divided by the number at risk as the time interval decreases. In the case of a hazard ratio, a value of 1 indicates that the relative risk is equal in both the first and second groups; a value greater or less than 1 indicates that the risk is greater in one group or another, depending on the inputs into the calculation.

Additionally, multiple threshold levels of expression may be determined. This can be the case in so-called “tertile,” “quartile,” or “quintile” analyses. In these methods, multiple groups can be considered together as a single population, and are divided into 3 or more bins having equal numbers of individuals. The boundary between two of these “bins” may be considered threshold levels of expression indicating a particular level of risk of a disease developing or signifying a physiological or cellular state. A risk may be assigned based on which “bin” a test subject falls into.

A subject includes any human or non-human mammal, including for example: a primate, cow, horse, pig, sheep, goat, dog, cat, or rodent, capable of developing cancer including human patients that are suspected of having cancer, that have been diagnosed with cancer, or that have a family history of cancer. Methods of identifying subjects suspected of having cancer include but are not limited to: physical examination, family medical history, subject medical history, endometrial biopsy, or a number of imaging technologies such as ultrasonography, computed tomography, magnetic resonance imaging, magnetic resonance spectroscopy, or positron emission tomography.

Cancer cells include any cells derived from a tumor, neoplasm, cancer, precancer, cell line, malignancy, or any other source of cells that have the potential to expand and grow to an unlimited degree. Cancer cells may be derived from naturally occurring sources or may be artificially created. Cancer cells may also be capable of invasion into other tissues and metastasis. Cancer cells further encompass any malignant cells that have invaded other tissues and/or metastasized. One or more cancer cells in the context of an organism may also be called a cancer, tumor, neoplasm, growth, malignancy, or any other term used in the art to describe cells in a cancerous state.

Examples of cancers that could serve as sources of cancer cells include solid tumors such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

Additional cancers that may serve as sources of cancer cells include blood borne cancers such as acute lymphoblastic leukemia (“ALL,”), acute lymphoblastic B-cell leukemia, acute lymphoblastic T-cell leukemia, acute myeloblastic leukemia (“AML”), acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), hairy cell leukemia, multiple myeloma, lymphoblastic leukemia, myelogenous leukemia, lymphocytic leukemia, myelocytic leukemia, Hodgkin's disease, non-Hodgkin's Lymphoma, Waldenstrom's macroglobulinemia, Heavy chain disease, and Polycythemia vera.

The present invention further provides kits to be used in assessing the expression of a RNA in a subject to assess the risk of developing disease. Kits include any combination of components that may facilitate the performance of an assay. A kit that facilitates assessing the expression of a RNA may include suitable nucleic acid-based and immunological reagents as well as suitable buffers, control reagents, and printed protocols.

Kits that facilitate nucleic acid based methods may further include one or more of the following: specific nucleic acids such as oligonucleotides, labeling reagents, enzymes including PCR amplification reagents such as Taq or Pfu, reverse transcriptase, or one or more other polymerases, and/or reagents that facilitate hybridization. Specific nucleic acids may include nucleic acids, polynucleotides, oligonucleotides (DNA, or RNA), or any combination of molecules that includes one or more of the above, or any other molecular entity capable of specific binding to a nucleic acid marker. In one aspect of the invention, the specific nucleic acid comprises one or more oligonucleotides capable of hybridizing to the marker.

A specific nucleic acid may include a label. A label may be any substance capable of aiding a machine, detector, sensor, device, or enhanced or unenhanced human eye from differentiating a sample that that displays positive expression from a sample that displays reduced expression. Examples of labels include but are not limited to: a radioactive isotope or chelate thereof, a dye (fluorescent or nonfluorescent,) stain, enzyme, or nonradioactive metal. Specific examples include but are not limited to: fluorescein, biotin, digoxigenin, alkaline phosphatase, biotin, streptavidin, ³H, ¹⁴C, ³²P, ³⁵S, or any other compound capable of emitting radiation, rhodamine, 4-(4′-dimethylaminophenylazo) benzoic acid (“Dabcyl”); 4-(4′-dimethylaminophenylazo)sulfonic acid (sulfonyl chloride) (“Dabsyl”); 5-((2-aminoethyl)-amino)-naphtalene-1-sulfonic acid (“EDANS”); Psoralene derivatives, haptens, cyanines, acridines, fluorescent rhodol derivatives, cholesterol derivatives; ethylenediaminetetraaceticacid (“EDTA”) and derivatives thereof or any other compound that signals the presence of the labeled nucleic acid. In one embodiment of the invention, the label includes one or more dyes optimized for use in genotyping. Examples of such dyes include but are not limited to: FAM, dR110, 5-FAM, 6FAM, dR6G, JOE, HEX, VIC, TET, dTAMRA, TAMRA, NED, dROX, PET, BHQ, Gold540, and LIZ.

An oligonucleotide may be any polynucleotide of at least 2 nucleotides. Oligonucleotides may be less than 10, less than 15, less than 20, less than 30, less than 40, less than 50, less than 75, less than 100, less than 200, less than 500, or more than 500 nucleotides in length. While oligonucleotides are often linear, they may, depending on their sequence and storage conditions, assume a two- or three-dimensional structure. Oligonucleotides may be chemically synthesized by any of a number of methods including sequential synthesis, solid phase synthesis, or any other synthesis method now known or yet to be disclosed. Alternatively, oligonucleotides may be produced by recombinant DNA based methods. In some aspects of the invention, an oligonucleotide may be 2 to 1000 bases in length. In other aspects, it may be 5 to 500 bases in length, 5 to 100 bases in length, 5 to 50 bases in length, or 10 to 30 bases in length. One skilled in the art would understand the length of oligonucleotide necessary to perform a particular task. Oligonucleotides may be directly labeled, used as primers in PCR or sequencing reactions, or affixed directly to a solid substrate as in oligonucleotide arrays among other things.

In some aspects of the invention, the probe may be affixed to a solid substrate. In other aspects of the invention, the sample may be affixed to a solid substrate. A probe or sample may be covalently bound to the substrate or it may be bound by some non covalent interaction including electrostatic, hydrophobic, hydrogen bonding, Van Der Waals, magnetic, or any other interaction by which a probe such as an oligonucleotide probe may be attached to a substrate while maintaining its ability to recognize the allele to which it has specificity. A substrate may be any solid or semi solid material onto which a probe may be affixed, attached or printed, either singly or in the formation of a microarray. Examples of substrate materials include but are not limited to polyvinyl, polysterene, polypropylene, polyester or any other plastic, glass, silicon dioxide or other silanes, hydrogels, gold, platinum, microbeads, micelles and other lipid formations, nitrocellulose, or nylon membranes. The substrate may take any form, including a spherical bead or flat surface. For example, the probe may be bound to a substrate in the case of an array. The sample may be bound to a substrate as (for example) the case of a Southern Blot, Northern blot or other method that affixes the sample to a substrate.

Kits may also contain reagents that detect proteins, often through the use of an antibody. These kits will contain one or more specific antibodies, buffers, and other reagents configured to detect binding of the antibody to the specific epitope. One or more of the antibodies may be labeled with a fluorescent, enzymatic, magnetic, metallic, chemical, or other label that signifies and/or locates the presence of specifically bound antibody. The kit may also contain one or more secondary antibodies that specifically recognize epitopes on other antibodies. These secondary antibodies may also be labeled. The concept of a secondary antibody also encompasses non-antibody ligands that specifically bind an epitope or label of another antibody. For example, streptavidin or avidin may bind to biotin conjugated to another antibody. Such a kit may also contain enzymatic substrates that change color or some other property in the presence of an enzyme that is conjugated to one or more antibodies included in the kit.

A kit may also contain an indication of a level of expression that signifies a particular physiological or cellular characteristic. An indication includes any guide to a level of expression that, using the kit in which the indication is provided, would signal the presence or absence of any physiological or cellular state that the kit is configured to detect. The indication may be expressed numerically, expressed as a color, expressed as an intensity of a band, derived from a standard curve, or derived from a control. The indication may be printed on a writing that may be included in the kit or it may be posted on the internet or embedded in a software package.

Where clinical applications are contemplated, it will be necessary to prepare pharmaceutical compositions that comprise expression vectors, virus stocks, proteins, antibodies or drugs in a form appropriate for the intended application. In many instances, this will entail preparing compositions that are essentially free of pyrogens, as well as other impurities that could be harmful to humans or animals. A pharmaceutical composition includes an active component such as Temozolomide, an inhibitor of a marker or other compound and a pharmacologically acceptable carrier. Pharmaceutically acceptable carriers include any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic or prophylactic compositions is contemplated. Supplementary active ingredients also can be incorporated into the compositions.

Pharmaceutical compositions include classic pharmaceutical preparations. Administration of these compositions according to the present invention will be via any common route so long as the marker tissue is available via that route. This includes oral, nasal, buccal, rectal, vaginal or topical. Alternatively, administration may be by orthotopic, intradermal, subcutaneous, intramuscular, intraperitoneal intratumoral, circumferentially, catheterization, or intravenous injection. Such compositions would normally be administered as pharmaceutically acceptable compositions. In some aspects of the invention, the pharmaceutical composition is formulated in such a way that it is capable of crossing the blood-brain barrier. However, in other aspects of the invention, the pharmaceutical composition may be administered directly to a tumor or placed in close proximity to a tumor.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations may contain a preservative to prevent the growth of microorganisms.

In some aspects of the invention, the cancer cells are derived from non-small cell lung cancer (NSCLC.) NSCLC includes any carcinoma derived from lung tissues that does not include small cell lung cancers. Examples of non-small cell lung cancers include adenocarcinomas, large cell carcinomas, and squamous cell carcinomas of the lung.

Expansion of a cancer cell includes any process that results in an increase in the number of individual cells derived from a cancer cell. Expansion of a cancer cell may result from mitotic division, proliferation, or any other form of expansion of a cancer cell, whether in vitro or in vivo. Expansion of a cancer cell further encompasses invasion and metastasis. A cancer cell may be in physical proximity to cancer cells from the same clone or from different clones that may or may not be genetically identical to it. Such aggregations may take the form of a colony, tumor or metastasis, any of which may occur in vivo or in vitro. Slowing the expansion of the cancer cell may be brought about either by inhibiting cellular processes that promote expansion or by bringing about cellular processes that inhibit expansion. Processes that inhibit expansion include processes that slow mitotic division and processes that promote cell senescence or cell death. Examples of specific processes that inhibit expansion include caspase dependent and independent pathways, autophagy, necrosis, apoptosis, and mitochondrial dependent and independent processes and further include any such processes yet to be disclosed.

In some aspects of the invention, inhibition of the expansion of the cancer cell is achieved through the use of an outside agent applied to the cancer cell for the purpose of slowing the expansion of the cancer cell. Such agents include natural or synthetic ligands, blockers, agonists, antagonists, or activators of receptors, immune cells such as CD8+ T cells, viruses, inhibitors of gene or protein expression such as siRNA or miR's, small molecules, pharmaceutical compositions, or any other composition of matter that when administered to the cancer cell would result in the slowing of the expansion of the cancer cell. The concept of agents that slow the expansion of a cancer cell encompasses restricting access to any natural or artificial agent necessary for cell survival including necessary nutrients, ligands, or cell-cell contacts. Examples of such agents and conditions include treatment with antiangiogenic inhibitors.

In some aspects of the invention, the agent that slows the expansion of the cancer cell comprises a tyrosine kinase inhibitor (TM). A tyrosine kinase catalyzes the transfer of a phosphate group to the tyrosine residue of a specific protein. If the tyrosine kinase inhibitor inhibits the action of a kinase necessary for expansion, differentiation or division of a cancer cell, expansion of the cancer cell will be slowed. A TKI includes any agent that inhibits the action of one or more tyrosine kinases in a specific or non-specific fashion. TKI's may include small molecules, antibodies, peptides, or anything that directly, indirectly, allosterically, or in any other way inhibits tyrosine residue phosphorylation. Specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-k1]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide (commonly known as sunitinib), 4-[4-[[4-chloro-3 (trifluoromethyl)phenyl]carbamoylamino]phenoxy]-N-methyl-pyridine-2-carboxamide (commonly known as sorafenib), EMD121974, and N-(3-ethynylphenyl)-6,7-bis(2-methoxyethoxy)quinazolin-4-amine (commonly known as erlotinib). In some aspects of the invention, the tyrosine kinase inhibitor has inhibitory activity upon the epidermal growth factor receptor (EGFR).

MicroRNA's (miR's) include non-coding RNA's 18 to 36 nucleotides, but preferably 21 to 25 nucleotides in length that inhibit gene expression by binding to a sequence complementary to the miR sequence, often located in the 3′ untranslated region (UTR) of the target mRNA. Mechanisms of gene silencing include repression of protein translation and downregulation of protein expression. The concept of miR's includes one or more non-nucleotide small molecule compositions of matter that may be derived from a parent miR that are capable of specifically binding the 3′ UTR a given gene, thereby silencing the expression of that gene.

MiR is readily detectable in blood and blood compartments such as serum or plasma or whole blood by any of a number of methods. See, for example, Chen X et al, Cell Research 18 983-984, October 2008; hereby incorporated by reference in its entirety.

MiR may be amplified by any of a number of techniques including reverse transcription followed by PCR. Some techniques of reverse transcription of miR use a targeted stem-loop primer to prime reverse transcription of the miR into a cDNA template. The cDNA template may then be used as a primer for any type of PCR including any type of quantitative PCR. A stem-loop oligonucleotide is a single stranded oligonucleotide that includes a sequence capable of binding to a specific marker because it includes a nucleic acid sequence complementary to the marker. The sequence complementary to the marker is flanked by inverted repeats that form self-complementary sequences. Such nucleotides may contain a fluorophore quencher pair at the 5′ and 3′ ends of the oligonucleotide. (See Buzdin and Lukyanov in Nucleic Acids Hybridization Modern Applications, pp 85-96, Springer 2007, hereby incorporated by reference in its entirety.)

### EXAMPLES

Elements and acts in the example are intended to illustrate the invention for the sake of simplicity and have not necessarily been rendered according to any particular sequence or embodiment. The example is also intended to establish possession of the invention by the Inventors.

### Example 1

Tyrosine kinase inhibitors (TKIs) are anticancer agents capable of specifically targeting tyrosine kinase receptors, which are often upregulated in certain cancer types. Erlotinib is a TKI that acts on the tyrosine kinase receptor EGFR and is frequently used in treating NSCLC. While erlotinib initially may be an effective treatment, tumors often acquire resistance to the TKI.

Erlotinib sensitivity was determined in the NSCLC cell line HCC827. An IC₅₀ value for erlotinib in HCC827 was determined using an AlamarBlue proliferation assay. One method of measuring IC₅₀ by AlamarBlue proliferation that may be used in determining an IC₅₀ is as follows:

Cells in the log phase of expansion are harvested and counted. The cells are then adjusted to a density of 1×10⁴ cells/ml, though the optimum cell density may vary between cell types. Cells other than untreated controls are then plated and exposed to erlotinib. The AlamarBlue® reagent is mixed, and then aseptically added to the cells in an amount around 10% of the well volume. Cells are incubated in the presence of the reagent from 4-8 hours. Cytotoxicity is measured using fluorescence spectrophotometry. Absorbance is measured at 570 nm and 600 nm blanked in a plate containing medium only. To calculate the percent difference in reduction between treated and control cells in Percentage difference between treated and control cells

$$\frac{( O 2 \times A 1 ) - ( O 1 \times A 2 ) \times 100}{( O 2 \times P 1 ) - ( O 1 \times P 2 )}$$

O1=molar extinction coefficient (E) of oxidized AlamarBlue® (Blue) at 570 nm*
O2=E of oxidized AlamarBlue® at 600 nm*
A1=absorbance of test wells at 570 nm
A2=absorbance of test wells at 600 nm
P1=absorbance of positive growth control well
(cells plus AlamarBlue® but no test agent) at 570 nm
30 P2=absorbance of positive growth control well
(cells plus AlamarBlue® but no test agent at 600 nm
Only one appropriate substitute wavelength may be used.

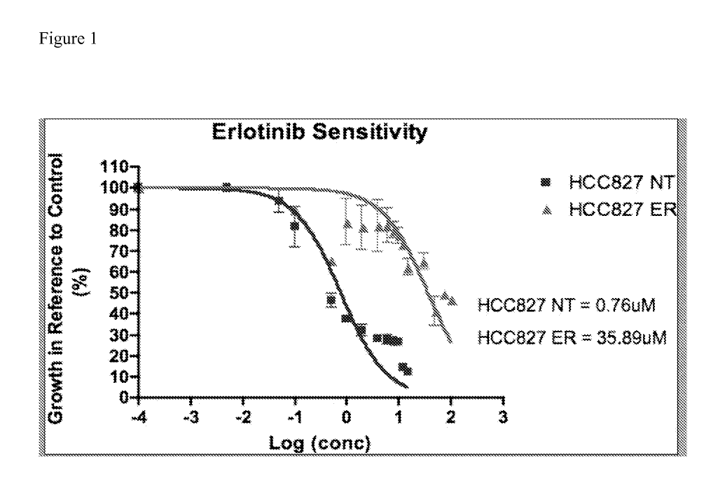

To create an erlotinib resistant cell line, HCC827 was exposed to ½ the IC₅₀ dose of erlotinib for 4 weeks (0.14 uM.) After a two week washout period with no drug treatment, TKI resistance was confirmed using an AlamarBlue assay. Referring now to FIG. 1, the sensitivity of the resistant cell line HCC827 ER (darker squares) to erlotinib was compared with the sensitivity of the parent line HCC827 (lighter triangles). As is apparent in FIG. 1, the IC₅₀ of the resistant cell line is nearly 50-fold higher than the IC₅₀ of the parent cell line. MiR microarray profiling was performed on the parent cell line (HCC827 NT) and the resistant cells (HCC827 ER) using a miR expression platform containing probes for 678 mature miRs and 451 human pre-miRs. Any differences in miR expression between HCC827 NT and HCC827 ER were analyzed by SAM analysis and validated by qRT-PCR. Referring now to Table 1, microarray profiling revealed that four miR's—miR-029a, let-7d, miR-100, and miR1250—were more highly expressed in the erlotinib resistant line relative to the parent line and five miR's—miR025, let-7i, miR-146a, miR-594 and miR-024 were less expressed in the resistant line relative to the parent line.

TABLE 1
miRNA microarray profiling results. Nine miRNA's were
significantly differentially expressed in erlotinib resistant
cells than the parent cell line.

| miRNA | Fold Change |
|---|---|
| hsa-miR-029a | 0.290 |
| hsa-let-7d | 0.376 |
| hsa-miR-100 | 0.476 |
| hsa-miR-1260 | 0.480 |
| hsa-miR-025 | 2.013 |
| hsa-let-7i | 2.082 |
| hsa-miR-146a | 2.090 |
| hsa-miR-594-Pre | 2.320 |
| hsa-miR-024 | 3.322 |

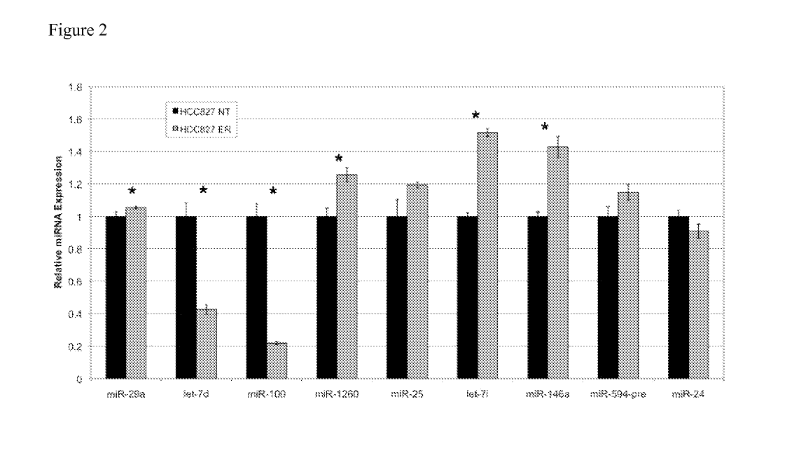

Referring now to FIG. 2, miR expression, when measured by qRT-PCR in parent (darker bars) and erlotinib resistant (lighter bars) cell lines show significant differences correlating with and validating the microarray data in let-7d, miR100, let-7i, and miR-146a when measured by qRT-PCR. An asterisk indicates a significant difference of p<0.05. The values in FIG. 2 were normalized to the ratio of hsa-miR-103 to hsa-miR-191, where miR-103 and miR-191 serve as normalizing miRs.

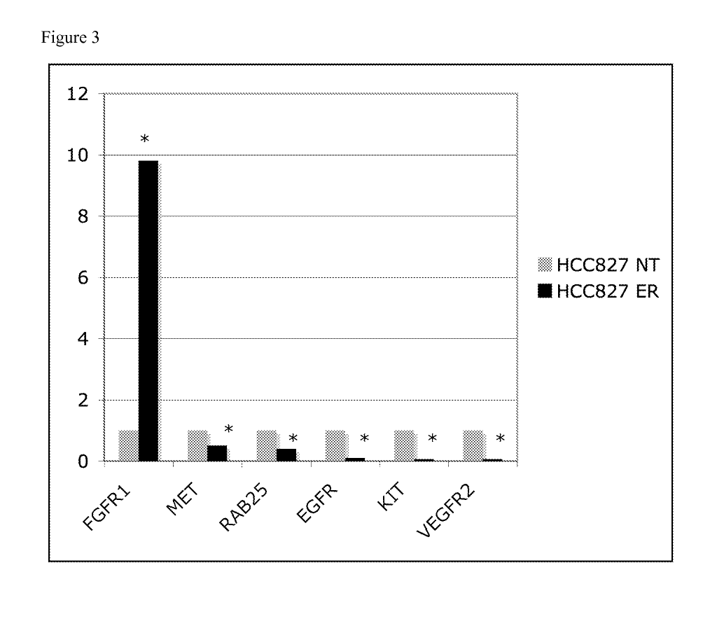

FGFR1 is overexpressed in the HCC827 ER line, while MET, RAB25, EGFR, and VEGFR2 are downregulated. Searching the 3′ UTRs for the miRNA seed sequences led to a number of potential miRNA regulators of these genes (see Table 2.) Referring now to FIG. 3, the mRNA expression of selected genes that have been implicated in the EGFR pathway was measured by qRT-PCR in HCC827 parent and HCC827 erlotinib resistant lines. Of the selected genes, FGFR1 was more highly expressed in the erlotinib resistant line relative to the parent line when normalized to expression controls (actin, GAPDH, and histone 3.3), while MET, RAB25, EGFR, and VEGFR2 were less expressed in the resistant line relative to the parent line when normalized to housekeeping controls (actin, GAPDH, and histone 3.3). An asterisk indicates a significant difference of p<0.05. Potential miRNA regulation of these genes was assessed by searching the 3′ UTRs of these genes for the miRNA seed sequences. qRT-PCR expression values are relative to HCC827 NT. Referring now to Table 2, mRNA regulation by miRNAs associated with TKI resistance was assessed by searching the 3′ untranslated regions (UTRs) for the miRNA seed sequences. Note that a seed sequence of let-7d, which is less expressed in the erlotinib resistant line is present in the 3′UTR of FGFR1; which is expressed in the resistant cell lines. This indicates that lower expression of let-7d is a marker of erlotinib resistance. Further, note that a seed sequence of let-7i, which is more highly expressed in the erlotinib resistant cells relative to an erlotinib-sensitive control, is present in the 3′UTR of MET and 3′ UTR of RAB25, and where MET and RAB25 are less expressed in the resistant cell lines. This indicates that higher expression of let-7i is a marker of erlotinib resistance.

TABLE 2
expression of genes in HCC827 ER relative to HCC827 parent line
including regulatory miR and their expression.

| | | | | Relative miR | |
|---|---|---|---|---|---|
| | | | | Expression | |
| | qRTPCR | | | in HCC827 ER | |
| Gene | Expression | miR | bp match | microarray | qRT-PCR |
| FGFR1 | 9.814 | let-7d | 4 × 6 bp | 0.376 | 0.425 |
| | | miR-1260 | 2 × 6 bp, | 0.480 | 1.256 |
| | | | 7 bp | | |
| | | miR-29a | 7 bp | 0.290 | 0.5 |
| MET | 0.513 | miR-25 | 7 bp, 6 bp | 2.013 | 1.193 |
| | | miR-24 | 7 bp | 3.332 | 0.909 |
| | | let-7i | 6 bp | 2.082 | 1.516 |
| RAB25 | 0.406 | let-7i | 6 bp | 2.082 | 1.516 |
| EGFR | 0.106 | miR-24 | 6 bp | 3.322 | 0.909 |
| | | miR-146a | 6 bp | 2.090 | 1.427 |
| KIT | 0.071 | miR-25 | 6 bp | 2.013 | 1.193 |
| | | miR-24 | 6 bp | 3.322 | 0.909 |
| | | let-7i | 6 bp | 2.082 | 1.516 |
| VEGFR2 | 0.063 | miR-24 | 6 bp | 3.322 | 0.909 |

### Example 2

TKI sensitivity was determined in NSCLC cell lines. Vandetanib and sunitinib sensitivity was measured in seven NSCLC cell lines. Erlotinib sensitivity was measured in nine NSCLC cell lines. An AlamarBlue proliferation assay at 72 hours was used to measure TKI sensitivity. GraphPad Prism 4.01 was used to determine IC₅₀ values. Expression of seven miRNAs was measured by qRT-PCR. qRT-PCR data were normalized to the reference RNU6 and fold changes were computed against the selected sensitive cell line for each drug. A two-sided z-test was used to compute the p-values for the fold changes. A score was calculated for each miRNA/drug combination to see whether a miRNA can determine resistance across cell lines. Fold changes were first ternarized and assigned a value of +1, −1, or 0. Elevated miRNA expression relative to control (fold change>1 AND p-value<0.05) was assigned a value of +1. Reduced miRNA expression relative to control (fold change<1 AND p-value<0.05) was assigned a value of −1. Any non-significant fold change (p-value>0.05) was assigned a value of 0. These values were then multiplied by a value of +1 or −1 as assigned by the resistance characteristics of the cell line: Resistant cell lines were assigned a value=+1. Sensitive cell lines were assigned a value=−1. The final score was computed by the sum of the points for the cells lines divided by the number of cell lines (the reference cell line was not used in computing the scores).

A “TKI resistance score” of “1” or “−1” suggests a perfect miRNA resistance indicator for the cell lines tested, while if the score is close to zero, it implies either too many insignificant values or too many inconsistencies. A positive score means the miRNA expression is lower in resistant cells as compared to sensitive cells, and the reverse is true of a negative score. The following cell lines were classified as sensitive for the respective TKI: For vandetanib, HCC827 (IC₅₀=0.55 μM) was classified as sensitive. For sunitinib, H1703 (IC₅₀=0.51 μM) was classified as sensitive. For erlotinib, HCC827 (IC₅₀=0.69 μM) and HCC4006 (IC₅₀=0.02 μM) were classified as sensitive. The remaining lines were classified as resistant for vandetanib, sunitinib, and erlotinib with IC₅₀s ranging from 2.1 to 13.9 μM, 4.8 to 14.2 μM, and 9.7 to >50 μM; respectively (see Table 3).

TABLE 3
IC₅₀ μM values for vandetanib, sunitinib, and erlotinib in NSCLC cell lines.

| | A549 | H358 | H460 | H520 | H1703 | H2122 | HCC827 | HCC4006 | H2073 |
|---|---|---|---|---|---|---|---|---|---|
| Vandetanib | 9.7 | 4.2 | 13.9 | 12.1 | 2.1 | 5.9 | 0.55 | ND | ND |
| Sunitinib | 5.5 | 14.2 | 5.6 | 8.2 | 0.51 | 4.8 | 8.7 | ND | ND |
| Erlotinib | >50 | 28.6 | 39.2 | >50 | >50 | 12.3 | 0.69 | 0.02 | 9.7 |

TKI sensitivity was measured using AlamarBlue proliferation assay.

Seven miRNAs were measured by qRT-PCR in the NSCLC cell lines. Applying the TKI resistance score, miR-21 and miR-424 had the best results for sunitinib with scores of 0.667 and −0.667, respectively. miR-424 had the best result for vandetanib and erlotinib with scores of 0.667 and 0.750 individually, and 0.714 across both groups together (see Table 4).

TKI Resistance Scores: Scores were assigned for each cell line/miRNA/TKI combination as described above. The further the score from zero, the more the miRNA was able to classify the cell line into the TKI resistant and TM sensitive group. Underlined scores indicate the miRNAs with the best ability to classify TM resistance in the NSCLC cell lines used in the study.

TABLE 4

| | miR-1308 | miR-21 | miR-29a | miR-29b | miR-29c | miR-30a−5p | miR-424 |
|---|---|---|---|---|---|---|---|
| V only | 0.333 | −0.500 | 0.333 | 0.500 | 0.167 | 0 | 0.667 |
| S only | 0 | 0.667 | −0.167 | 1.67 | 0 | 0 | −0.667 |
| E only | 0.250 | −0.250 | 0.250 | 0.375 | 0.125 | 0 | 0.750 |
| V + S | 0.167 | 0.083 | 0.083 | 0.333 | 0.083 | 0 | 0 |
| V + E | 0.286 | −0.357 | 0.286 | 0.429 | 0.143 | 0 | 0.714 |
| S + E | 0.143 | 0.143 | 0.071 | 0.286 | 0.071 | 0 | 0.143 |
| V + S + E | 0.200 | −0.050 | 0.150 | 0.350 | 0.100 | 0 | 0.300 |

V = Vandetanib, S = Sunitinib, E = Erlotinib.

There are varying in vitro sensitivities across TKIs for a currently FDA approved TKI (erlotinib) or TKIs current being tested in NSCLC clinical trials (vandetanib and sunitinib). NSCLC lines have similar sensitivity and resistance to vandetanib and erlotinib, although vandetanib appears to be more potent in vitro, as reflected by lower IC₅₀ values. There are miRNAs associated with TM resistance. miR-424 is a marker for both vandetanib and erlotinib resistance, while miR-21 is a marker for sunitinib resistance.

### Example 3

Tyrosine kinase inhibitors (TKIs) are anticancer agents capable of specifically targeting tyrosine kinase receptors (TKR) often up-regulated in certain cancers. Sunitinib is a TM acting on multiple TKRs including VEGFR, PDGFRβ, and FLT3. Sunitinib is approved for the treatment of advanced kidney and GIST cancers and is currently in clinical trials in other solid tumors, including non-small cell lung cancer (NSCLC). We sought to identify in vitro microRNA (miRNA) biomarkers associated with sunitinib resistance (SR) in NSCLC cell lines.

### Methods:

Alamar blue proliferation assays at 72 hours were used to determine sunitinib sensitivity in a panel of seven NSCLC cell lines. GraphPad Prism 4.01 was used to calculate IC₅₀ values. The sensitive NSCLC cell line H1703 was used to create sunitinib resistant cells by exposing three independent sets of cells to sunitinib over several weeks. mRNA microarray profiling was performed on the three H1703 sunitinib resistant cell lines (SRa, b, and c) and the control H1703 cells. GenoSensor GenoExplorer™ microRNA Expression System (Sanger miRNA Registry version 14 containing probes for 904 mature miRNAs and 450 pre-miRNAs) was used to screen miR expression. Differences in miRs expression between the parent and resistant cells were identified using at test with Benjamini-Hochberg correction and validated by qPCR. A score was calculated for each miRNA to determine whether a miRNA can predict SR.

TKI Score Calculation: QPCR fold changes were first ternarized and assigned a value of +1, −1, or 0: Significant increase (fold change>1 AND p-value<0.05)=+1. Significant decrease (fold change<1 AND p-value<0.05)=−1; Non-significant fold change (p-value>0.05)=0. These values were then multiplied by a value of +1 or −1 as assigned by the resistance characteristics of the cell line: Resistance cell lines=+1; Sensitive cell line=−1. The final score is computed by the sum of the points for the cells lines divided by the number of cell lines (reference cell line was not used in computing the scores). A “TKI resistance score” of “1” or “−1” suggests a perfect miRNA resistance indicator for the cell lines tested, while if the score is close to zero, it implies either too many insignificant values or too many inconsistencies. A positive score means the miRNA expression is increased in resistant cells as compared to sensitive cells, and the reverse is true of a negative score.

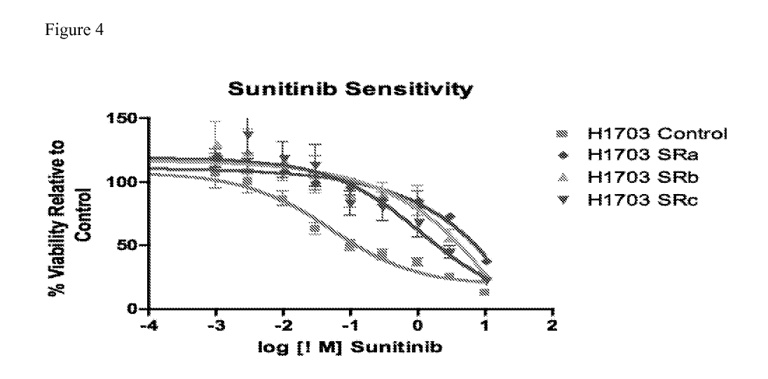

In FIG. 4, H1703 cells with acquired sunitinib resistance were subjected to Alamar blue assays in different concentrations of Sunitinib. The H1703 Control has an IC₅₀ of 0.13 μM. H1703 Sunitinib resistant line A (H1703 SRa) has an IC₅₀ of 7.6 μM. Sunitinib resistant line B H1703 H1703 SRb has an IC₅₀ of 4.37 μM. Sunitinib resistant line C(H1703 SRc) has an IC₅₀ of 2.09 μM.

Eighteen miRNAs were up-regulated in H1703 SR cells by >9-fold, and one miRNA was down-regulated in H1703 SR cells by >9 fold. These four miRNAs were chosen for qPCR validation because of previous reports of their importance in cancer. The results are listed in

TABLE 5
miRNA microarray results from H1703 control cells and
H1703 SR cells.

| | p-value | BH p-value | q-value | FDR | Fold Change |
|---|---|---|---|---|---|
| hsa-miR-21 | 0.0002 | 0.0013 | 0.0005 | 0.0005 | 24.75 |
| hsa-miR-29b | 0.0066 | 0.0158 | 0.0029 | 0.0029 | 13.07 |
| hsa-miR-23a | 0.0092 | 0.0195 | 0.0031 | 0.0031 | 11.49 |
| hsa-miR-23b | 0.0078 | 0.0182 | 0.0031 | 0.0023 | 9.23 |

BH-Benjamin-Hochberg,
FDR = False Discovery Rate.

TABLE 6
TKI resistance scores

| | miR-21 | miR-23a | miR-23b | miR-29b |
|---|---|---|---|---|
| Group A | −0.33 | 0.67 | 1.00 | 0.00 |
| Group B | 0.67 | −1.00 | 0.50 | 0.67 |
| Group C | 0.33 | −0.44 | 0.67 | 0.44 |

Scores were based on qPCR miRNA expression.
RNU6 was used as the reference RNA
Group A = H1703, H1703 Sra, SRb, and SRc.
Group B = panel of seven NSCLC cell lines.
Group C = panel of NSCLC cell lines and three H1703 SR lines.

TABLE 7
Sunitinib IC₅₀ measurements and miRNA expression in NSCLC
cell lines. Sunitinib IC₅₀ experiments were repeated three times
and the mean value shown. miRNA expression was by qPCR.

| | Sunitinib | | | | |
|---|---|---|---|---|---|
| Cell Line | IC₅₀(μM) | miR-21 | miR-23a | miR-23b | miR-29b |
| H1703 | 0.130 | 1 | 1 | 1 | 1 |
| H1703 SRa | 7.6 | 0.76 | 1.25 | 2.09* | 3.44* |
| H1703 SRb | 4.37 | 0.49* | 2.40* | 2.43* | 1.15* |
| H1703 SRc | 2.09 | 1.23 | 2.27* | 4.50* | 2.71 |
| H2122 | 4.8 | 5.79* | 0.43* | 0.94 | 1.70* |
| A549 | 5.5 | 12.33* | 0.60* | 9.29* | 2.24* |
| H460 | 5.6 | 0.66 | 0.22* | 0.45* | 0.72* |
| H520 | 8.2 | 0.26 | 0.04* | 1.03* | 0.08* |
| HCC827 | 8.7 | 9.77* | 0.22* | 0.60* | 0.55* |
| H358 | 14.2 | 12.87* | 0.38* | 2.43* | 1.55* |

*significantly different expression from H1703 (sensitive) cell line.

### Summary:

Sunitinib sensitivity was determined in seven NSCLC cell lines. (Sensitive=H1703; Resistant=H2122, A549, H460, HCC827, H520, H358). Sunitinib resistant H1703 cells (H1703 SRa, SRb, and SRc) were successfully created. miRNA profiling identified up-regulation of miR-21, miR-23a, miR-23b, and miR-29b in the H1703 SR cells. TKI resistance scores based on qPCR data revealed miR-23b to be the best indicator of sunitinib resistance having a score of 1 when analyzing only the H1703 SR lines and a score of 0.67 when analyzing the H1703 SR cell lines together with the other six NSCLC cell lines.

### REFERENCES

So as to reduce the complexity and length of the Detailed Specification, Inventor herein expressly incorporates by reference to the extent allowed all of the following materials.
1. Calin G A et al, Proc Natl Acad Sci USA 99, 15524-15529 (2002).
2. Kumar M S et al, Nat Genet. 39, 673-677 (2007).
3. Eder M and Scherr M, N Engl J Med; 352, 2446-2448 (2005).
4. Scott G K et al, J Biol Chem 282, 1479-1486 (2007).
5. Calin G A, et al. Proc Natl Acad Sci USA; 101 (32):11755-11760 (2004).
6. Lu J et al. Nature 435 (7043):834-838. (2005).
7. Brown et al. Publication Number US 2007/0161004, filed May 2005.

I claim:

1. A method of classifying a subject into a cohort comprising:
receiving a sample from a subject and isolating RNA from the sample;
adding a first reagent capable of specific binding to a marker including a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, and SEQ ID NO. 15 to a mixture comprising the sample; and
subjecting the mixture to conditions that allow detection of the binding of the first reagent to the marker;
wherein the subject is suspected of having non-small cell lung cancer, wherein the cohort comprises two or more individuals unlikely to respond to treatment with a tyrosine kinase inhibitor and wherein the tyrosine kinase inhibitor is selected from the group consisting of erlotinib, sunitinib, and vandetanib.

2. The method of claim 1 wherein the first reagent comprises a first oligonucleotide.

3. The method of claim 2 wherein the first oligonucleotide comprises a stem-loop oligonucleotide.

4. The method of claim 2 further comprising adding reverse transcriptase to the mixture and wherein the conditions comprise allowing the formation of a DNA template comprising the marker.

5. The method of claim 4 further comprising adding a second oligonucleotide and a third oligonucleotide to the mixture, wherein the second oligonucleotide and the third oligonucleotide bind to opposite strands of the DNA template and wherein the conditions comprise nucleic acid amplification.

6. The method of claim 5 wherein the second oligonucleotide is capable of binding to the 5′→3′ strand of the cDNA template.

7. The method of claim 5 further comprising adding a fourth oligonucleotide to the mixture wherein the fourth oligonucleotide binds to the DNA template between the sequences to which the second oligonucleotide and the third oligonucleotide are capable of binding.

8. The method of claim 7 wherein the fourth nucleic acid comprises a label.

9. The method of claim 8 wherein the label comprises a fluorescent label.

10. The method of claim 9 wherein the fluorescent compound is selected from the group consisting of FAM, dR110, 5-FAM, 6FAM, dR6G, JOE, HEX, VIC, TET, dTAMRA, TAMRA, NED, dROX, PET, BHQ, Gold540, and LIZ.

11. The method of claim 4 wherein the conditions comprise DNA sequencing.

12. The method of claim 1 wherein the first reagent is affixed to a substrate.

13. The method of claim 1 wherein the sample comprises serum.

14. The method of claim 1 wherein the sample comprises a cell.

15. The method of claim 14 wherein the sample comprises a lung biopsy.

16. The method of claim 14 wherein the sample comprises a metastatic tumor.

17. The method of claim 1 further comprising collecting a sample from the subject.

18. The method of claim 1 wherein the marker includes a sequence selected from the group consisting of SEQ ID NO. 1, SEQ ID NO. 2, SEQ ID NO. 3, SEQ ID NO. 4, SEQ ID NO. 6, SEQ ID NO. 10, SEQ ID NO. 11, SEQ ID NO. 12, SEQ ID NO. 13, SEQ ID NO. 14, and SEQ ID NO. 15 and wherein the tyrosine kinase inhibitor comprises erlotinib.

19. The method of claim 1 wherein the marker includes a sequence selected from the group consisting of SEQ ID NO. 5, SEQ ID NO. 6, SEQ ID NO. 7, SEQ ID NO. 8, and SEQ ID NO. 9 and where the tyrosine kinase inhibitor comprises sunitinib.

20. The method of claim 1 wherein the marker includes a sequence selected from the group consisting of SEQ ID NO. 6 and wherein the tyrosine kinase inhibitor comprises vandetanib.

21. A kit used to classify a subject into a cohort comprising:
a first reagent capable of specific binding to a marker that includes a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, and SEQ ID NO. 15; and
an indication of a result that signifies classification of the subject into the cohort wherein the cohort comprises two or more individuals unlikely to respond to treatment with a tyrosine kinase inhibitor and wherein the tyrosine kinase inhibitor is selected from the group consisting of erlotinib, sunitinib, and vandetanib.

22. The kit of claim 21 wherein the first reagent comprises a first oligonucleotide.

23. The kit of claim 22 wherein the first oligonucleotide is a stem loop oligonucleotide.

24. The kit of claim 21 further comprising a second oligonucleotide and a third oligonucleotide wherein the second oligonucleotide and the third oligonucleotide are capable of binding to opposite strands of a DNA construct comprising the reverse transcription product of the marker.

25. The kit of claim 24 wherein the second oligonucleotide is capable of binding to the 5′→3′ strand of the DNA construct.

26. The kit of claim 24 further comprising a fourth oligonucleotide capable of binding to a sequence between the sequences to which the second oligonucleotide and the third oligonucleotide are capable of binding.

27. The kit of claim 26 comprising a label.

28. The kit of claim 27 wherein the label comprises a fluorescent label.

29. The kit of claim 28 wherein the fluorescent label is selected from the group consisting of FAM, dR110, 5-FAM, 6FAM, dR6G, JOE, HEX, VIC, TET, dTAMRA, TAMRA, NED, dROX, PET, and LIZ.

30. The kit of claim 21 further comprising an enzyme.

31. The kit of claim 30 wherein the enzyme comprises a DNA polymerase.

32. The kit of claim 31 wherein the DNA polymerase is a thermostable DNA polymerase.

33. The kit of claim 30 wherein the enzyme comprises a reverse transcriptase.

34. The kit of claim 21 wherein the first reagent is affixed to a substrate.

35. The kit of claim 21 further comprising a device to be used in collecting a sample.

36. The kit of claim 21 wherein the result comprises a ΔCt value.

37. The kit of claim 21 wherein the result comprises nucleic acid sequence data.

38. The kit of claim 21 wherein the indication comprises a positive control.

39. The kit of claim 21 wherein the indication comprises a writing.

40. The kit of claim 39 wherein the writing is physically included in the kit.

41. The kit of claim 39 wherein the writing is made available via a website.

42. The kit of claim 39 wherein the writing comprises an amplification plot.

43. The kit of claim 39 wherein the writing comprises a photograph.

44. The kit of claim 39 wherein the indication comprises software configured to detect result as input and classification of the subject into the cohort as output.

45. The kit of claim 44 wherein the software is incorporated into a machine configured to detect fluorescence.

46. A method of treating a subject comprising:
receiving a sample from a subject and isolating RNA from the sample;
adding a first reagent capable of specific binding to a marker including a sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, and SEQ ID NO. 15 to a mixture comprising the RNA;
subjecting the mixture to conditions that allow detection of the binding of the first reagent to the sequence; and
treating with a tyrosine kinase inhibitor based upon a result indicated by the binding of the first reagent to the sequence;
wherein the subject is suspected of having non-small cell lung cancer, the cohort comprises two or more individuals likely to respond to treatment with a tyrosine kinase inhibitor and wherein the tyrosine kinase inhibitor is selected from the group consisting of erlotinib, sunitinib, and vandetanib.

47. The method of claim 46 wherein the first reagent comprises a first oligonucleotide.

48. The method of claim 47 wherein the first oligonucleotide comprises a stem-loop oligonucleotide.

49. The method of claim 48 further comprising adding reverse transcriptase to the mixture and wherein the conditions comprise allowing the formation of a DNA template comprising the marker.

50. The method of claim 49 further comprising adding a second oligonucleotide and a third oligonucleotide to the mixture, wherein the second oligonucleotide and the third oligonucleotide bind to opposite strands of the DNA template and wherein the conditions comprise nucleic acid amplification.

51. The method of claim 50 wherein the second oligonucleotide is capable of binding to the 5′ 3′ strand of the cDNA template.

52. The method of claim 50 further comprising adding a fourth oligonucleotide to the mixture wherein the fourth oligonucleotide binds to the cDNA template between the sequences to which the second oligonucleotide and the third oligonucleotide are capable of binding.

53. The method of claim 52 wherein the fourth nucleic acid comprises a label.

54. The method of claim 53 wherein the label comprises a fluorescent label.

55. The method of claim 54 wherein the fluorescent compound is selected from the group consisting of FAM, dR110, 5-FAM, 6FAM, dR6G, JOE, HEX, VIC, TET, dTAMRA, TAMRA, NED, dROX, PET, BHQ, Gold540, and LIZ.

56. The method of claim 49 wherein the conditions comprise DNA sequencing.

57. The method of claim 46 wherein the first reagent is affixed to a substrate.

58. The method of claim 46 wherein the sample comprises serum.

59. The method of claim 46 wherein the sample comprises a cell.

60. The method of claim 59 wherein the sample comprises a lung biopsy.

61. The method of claim 59 wherein the sample comprises a metastatic tumor.

62. The method of claim 46 further comprising collecting a sample from the subject.

63. The method of claim 46 wherein the marker includes a sequence selected from the group consisting of SEQ ID NO. 1, SEQ ID NO. 2, SEQ ID NO. 3, SEQ ID NO. 4, SEQ ID NO. 6, SEQ ID NO. 10, SEQ ID NO. 11, SEQ ID NO. 12, SEQ ID NO. 13, SEQ ID NO. 14, and SEQ ID NO. 15 and wherein the tyrosine kinase inhibitor comprises erlotinib.

64. The method of claim 46 wherein the marker includes a sequence selected from the group consisting of SEQ ID NO. 5, SEQ ID NO. 6, SEQ ID NO. 7, SEQ ID NO. 8, and SEQ ID NO. 9 and where the tyrosine kinase inhibitor comprises sunitinib.

65. The method of claim 46 wherein the marker includes a sequence selected from the group consisting of SEQ ID NO. 6 and wherein the tyrosine kinase inhibitor comprises vandetanib.

66. The method of claim 46 wherein classifying the subject into a group is performed on the recommendation of a writing.

67. The method of claim 66 wherein the writing is affixed to a container holding the tyrosine kinase inhibitor.

68. The method of claim 46 wherein the result comprises a ΔCt value.

69. The method of claim 46 wherein the result comprises a nucleic acid sequence data.
